(19) United States
(12) Patent Application Publication
(10) Pub. No.: US 2008/0031401 A1
(43) Pub. Date: Feb. 7, 2008

(54) Method and apparatus for displaying a region to be examined of an examination object

(76) Inventors: Jan Boese, Eckental (DE); Marcus Pfister, Bubenreuth (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/704,425

(22) Filed: Feb. 8, 2007

(30) Foreign Application Priority Data
Feb. 9, 2006 (DE) 10 2006 006 041.5

(51) Int. Cl. A61B 6/03

(52) U.S. Cl. 378/4

(57) ABSTRACT

The invention relates to a method and an apparatus for spatially displaying a region to be examined of an examination object, which has an x-ray emitter which can be rotated about the examination object in order to generate an x-ray beams and an x-ray detector which can be rotated about the examination object in order to record a part of the x-ray beam penetrating the examination object, with a facility having an opening for fading-in a split beam being arranged at a distance from the x-ray emitter. By being able to adjust the opening and/or the distance of the fade-in facility, a method and an apparatus can be provided, with which the radiation exposure for an examination object is reduced.

### CROSS REFERENCE TO RELATED APPLICATIONS

This application claims priority of German application No. 10 2006 006 041.5 filed Feb. 9, 2006, which is incorporated by reference herein in its entirety.

### FIELD OF THE INVENTION

The present invention generally relates to improvements in x-ray devices, in particular in the field of medical technology. The invention specifically relates to a method and an apparatus for spatially displaying a region to be examined of an examination object.

### BACKGROUND OF THE INVENTION

Conventional x-ray systems remain an important instrument for medical diagnosis and patient monitoring irrespective of progress made within the field of medical technology, particularly imaging methods, e.g. computed tomography and magnetic resonance tomography. X-ray examinations can be used in diagnostics, e.g. for clearly displaying bone fractures, tumors, cysts, calcifications, trapped air or also for preventive medical examinations. On the other hand, x-ray systems can also be used fluoroscopically, e.g. with angiographic examinations for recording the vascular system of a patient, controlling medical interventions, locating medical instruments etc. By reducing the radiation dose used for patients for x-ray examinations, in particular by means of technical advances, further areas of application are developed for x-ray technology, in particular for angiography systems used interventionally.

Modern angiography systems, for instance Siemens Axion Artis, not only allow two-dimensional images of a patient to be obtained, but by recording a number of images and/or projection data sets for the same examination region from different recording directions, spatial displays of an examination region can also be determined. A single recording journey is sufficient for native recordings of an examination region, e.g. an organ in its anatomical environment without the supply of contrast agents. In this way, a C-arm rotates for instance about the examination region, with image data sets from the examination region being recorded during the journey. A reverse projection allows a spatial display of the examination region to be determined from the recorded images. However, an increased radiation exposure for the examination object results from the plurality of recordings required in order to determine a spatial display.

The published patent application 10 2004 016 586 A1 discloses an image reconstruction device for an x-ray apparatus as well as a method for local 3D reconstruction of an object region of an examination object from 2D image data of a number of 2D fluoroscopic images of the examination object, which were recorded using the x-ray apparatus in chronological order with differently known projection geometries. The method and the image reconstruction device enable a 3D image reconstruction of a moving, locally-limited, object region without movement artifacts, in a simple manner.

If the aim of the examination is to display subtraction images, a number of recording journeys are required in order to produce these. A mask image is generally first recorded in order to generate a subtraction image, said mask image corresponding to a native recording of the region of the examination object of interest. Finally, a recording of the same region is carried out by supplying a contrast agent. If these images are subtracted from one another, a subtraction image is achieved. Spatial displays can likewise be generated from subtraction images of this type, if subtraction images are recorded for a number of projection directions.

The spatial displays which can be determined today by means of angiography systems sometimes achieve the quality of spatial displays, which are obtained by means of computed tomography. The surface of an x-ray beam projected and radiated in this manner in order to determine spatial displays generally amounts to approximately 400 cm² or 20 cm by 20 cm, whereas the region x-rayed in the case of computed tomography is generally limited to a few square millimeters per revolution.

If interventions are carried out on critical parts of the body, such as neurolysis, biopsies of parenchymatous tissue, drainage treatment for pathological fluid accumulation, radiological, interventional pain therapy, TIPSS—Transjugular Intrahepatic PortoSystemic Shunt, percutanous bile duct and biliary tract drainage, further special therapies, e.g. radio frequency ablation etc., spatial displays for the improved control of interventions, e.g. the insertion of a thin puncture needle into the critical part of the body, are desired. To this end, a spatial display of the relevant examination region is determined between two movements of the medical instrument, using an inserted instrument. The process of inserting the needle and puncturing the tissue can thus be monitored for instance.

Until now, the control of medical interventions which is currently based on 3D imaging and/or spatial displays, said interventions requiring a low contrast resolution and/or accurate information relating to the spatial position of an instrument, e.g. a needle, in the body, has generally been carried out using computed tomography. A layer or a few thin layers of the examination object is recorded here, and a spatial display of the examined region is reconstructed. The disadvantages of the computed tomography method result from poor patient accessibility, which is restricted for the medical personnel during the image recording of a cylinder-shaped surface, and an increased radiation exposure for patients, as the option of fluoroscopy, i.e. recording a two-dimensional projection using a low x-ray dose, does not exist here.

### SUMMARY OF THE INVENTION

The object underlying the invention is to provide a method and an apparatus, with which a spatial display for a region of an examination object is determined with a reduced radiation exposure compared with conventional x-ray apparatuses and computed tomographs. Furthermore, the examination object should be easily accessible for medical personnel while determining the spatial display.

The method spatially displays a region to be examined of an examination object, with a plurality of two-dimensional projection data sets characterized in each instance by a projection direction being recorded, with a projection data set being obtained from an x-ray beam penetrating the examination object, said x-ray beam comprising a beam centerline extending in the projection direction and being restricted by a beam limiting surface, and with the beam centerline of the plurality of projection data sets lying in a common examination plane penetrating the region to be examined, with a three-dimensional image data set of the region to be examined being determined and displayed from the projection data sets by means of an image reconstruction method. The apparatus spatially displays a region to be examined of an examination object, which apparatus has an x-ray emitter which can be rotated about the examination object in order to generate an x-ray beam and an x-ray detector which can be rotated about the examination object in order to record a part of the x-ray beam penetrating the examination object, with a fade-in facility with an aperture for fading-out a split beam being arranged at a distance from the x-ray emitter.

The part of the object to be achieved by the method is achieved with a generic method of the type mentioned at the start such that the x-ray beam is adjusted as a function of the region to be examined such that the beam limiting surface closely surrounds the region to be examined. In this way, the examination object is generally positioned on a support such that the region to be examined is recorded by an x-ray beam, which emanates from an x-ray source. The region to be examined of the examination object is defined by the medical person skilled in the art. He/she is to make a selection such that the radiation exposure for the examination object is as minimal as possible and a high-quality spatial display of the region to be examined of the examination object, e.g. precisely one organ, is enabled for the medical person skilled in the art.

The region to be examined is recorded in two-dimensional form by means of an x-ray beam adjusted to the region to be examined. The x-ray beam is partially absorbed when penetrating the examination object. The transmitted part of the x-ray beam is detected using an x-ray detector. Digital flat-screen detectors are generally required to resolve low image contrasts. A beam limiting surface of the x-ray beam separates the x-ray beam from the x-ray beam-free environment. The signal detected over the detector surface represents a projection of the region to be examined of the examination object. A projection data set is recorded in a specific direction, the projection direction, which is predetermined by the direction of the central beam of the x-ray beam. The direction of the central beam of the x-ray beam penetrating the region to be examined is referred to as a beam centerline. To be able to determine a spatial display of the region to be examined, it is necessary to x-ray the region to be examined from different projection directions. This can be carried out for instance by rotating a recording facility of the apparatus for spatial resolution about the region to be examined. By rotating the recording facility, a plurality of projection data sets in different projection directions of the region to be examined is achieved, from which a data set for spatially displaying the region to be examined is determined by means of reconstruction algorithms. The spatial display is generally shown on an output unit.

Since, in order to determine a spatial display of a region to be examined, a plurality of projection data sets is recorded by x-raying a region to be examined, the radiation exposure for the examination object is generally increased in comparison with simple, two-dimensional displays of the examination object determined with a low x-ray dose, i.e. the classical fluoroscopy method. To reduce the radiation exposure while determining a three-dimensional display, the beam limiting surface of the x-ray beam, according to the invention, is adjusted to the region to be examined. To this end, the shape of the x-ray beam through a fade-in facility can be of any design. The beam limiting surfaces are adjusted here such that the borders of the region to be examined are surrounded as closely as possible by the beam limiting surfaces. This can be carried out section by section for the region to be examined, i.e. the close enclosure consists in the tangency and/or contact of as many border points of the region to be examined as possible, or for the whole boundary of the region to be examined. If the region to be examined features different dimensions in different projection directions, the beam limiting surface can be adjusted to the region to be examined for each projection direction.

A close enclosure can also exist in maintaining a specific, minimal distance of the beam limiting surface from the region to be examined. By adjusting the x-ray beam to the region to be examined, it is possible to reduce the radiation exposure, adjusted for instance to age, size and sex of a patient, compared to conventional x-ray examinations, which serve to determine a spatial display. The x-ray beam and/or the beam limiting surface can be adjusted to the region to be examined for each projection direction, as a function of the selected shape of x-ray beam. By adjusting the beam limiting surface, so that this closely surrounds the region to be examined, the calculation time for reconstructing the spatial display can also be reduced, as the region to be reconstructed reduces in size. In addition, the use of open x-ray diagnosis device allows the examination object to be easily accessible for the medical personnel, and thereby assists with interventions on the examination object.

In an advantageous embodiment of the invention, the x-ray beam is wedge-shaped, so that two essentially flat partial areas of the beam limiting surface inclined towards one another rest closely against the region to be examined. The partial areas inclined towards one another are designed as plane segments. The further beam limiting surfaces can be of any design. A wedge-shaped x-ray beam thus allows the radiated surface of the examination object to be restricted in a specific direction. This restriction preferably takes place in the head/foot direction of a human patient. In this way, the region to be examined is usually only closely surrounded section by section, i.e. tangented piece by piece. In the non-restricted direction, the examination object can be x-rayed such that the width of the x-ray detector is fully utilized. A restriction of this type of the x-rayed surface in the head/foot direction also prevents reconstruction artifacts. The wedge-shaped x-ray beam is advantageous particularly in the field of child radiology and/or fluoroscopy, as the x-rayed region of the examination object can be adjusted here to the anatomy of the patient in a simple manner. A radiation exposure which is reduced in comparison with conventional 3D x-ray examinations can thus be achieved.

In a further advantageous embodiment of the invention, the x-ray beam is designed in the shape of a pyramid, so that two pairs of essentially flat partial areas of the beam limiting surface which are inclined towards one another rest closely against the region to be examined. In this way, the two pairs of partial surfaces which are inclined towards one another are designed in each instance as sections of planes. An x-ray beam shape of this type allows the x-rayed region to be restricted in two directions, independent of one another. The x-ray beam can thus be adjusted to the region to be examined in a more effective manner than a wedge-shaped x-ray beam, particularly if the medical person skilled in the art would like to determine a spatial display of an individual organ or organ parts within their more immediate environment. The radiation exposure for the examination object can thus be further reduced.

In a preferred embodiment of the invention, all beam centerlines of an examination plane run through a common examination center lying in the region to be examined. By rotating the adjusted x-ray beam about the region to be examined, the projection directions of different projection data sets intersect in an examination center, which is also referred to as an isocenter. The isocenter of the examination is preferably positioned in the center of the region to be examined. By rotating the adjusted x-ray beam about the examination object, a plurality of two-dimensional projection data sets of different projection directions of the region to be examined are recorded using an x-ray apparatus of a simple design, e.g. a monoplane C-arm x-ray device. Alternatively, a biplane C arm x-ray device can also be used.

In a further preferred embodiment of the invention, the projection data sets of an examination plane are recorded with a constant beam setting. To this end, the x-ray beam is adjusted such that the region to be examined can be recorded for all projection directions using a constant beam setting. A constant beam setting for different projection directions reduces the effort required to implement the method, since a control of the beam setting, manually or automatically, is not necessary as a function of the projection direction. This possibly also reduces the examination time. A disadvantage here is however that the examination object is subjected to an increased radiation exposure.

The part of the object to be achieved with this apparatus is achieved with a generic apparatus of the type mentioned at the start, such that the aperture and/or the distance of the fade-in facility can be adjusted. The aperture can be of any design, e.g. as a circular area, an ellipsoid area or in other conceivable, also non-symmetrical surface shapes, with the size of the aperture being adjustable. This can be enabled for instance with a circular diaphragm by means of an iris diaphragm. The x-ray beam emitted by the x-ray emitter is divided into two parts by means of the fade-in facility. The faded-in part of the x-ray beam passes through the aperture; the faded-out part is reflected or absorbed at the diaphragm elements restricting the x-ray beam. Diaphragm elements having x-ray-absorbent characteristics are produced for instance from lead or other elements exhibiting high atomic numbers. The use of reflection can be carried out by means of reflectors, for example by utilizing Bragg conditions of monocrystalline material layers adjusted to the wavelengths.

A number of advantageously adjustable apertures can be arranged on a common facility, a diaphragm disk for example, in order to be able to rapidly select the most suitable aperture in each instance for the region to be examined. Furthermore, the distance of the aperture from the x-ray source can be adjusted by means of regulating means, designed for instance as a telescope arm. The fade-in facility is preferably arranged in a moveable fashion in respect of the x-ray source, while the x-ray source is fixed in respect of the x-ray emitter. By changing the distance of the aperture from the x-ray emitter, the beam can likewise be adjusted.

In an advantageous embodiment of the invention, the aperture is designed as a slit. The aperture is predetermined by the width of the slit and can be adjusted to the region to be examined by displacing moveably arranged diaphragm elements restricting the x-ray beam. As, in the case of a slit, the slit width can only be adjusted in one direction, the x-ray beam in the directions not restricted by the diaphragm elements is, if necessary, adjusted to the spatial expansion of the used detector. The slit is preferably arranged in parallel to rotary motions of the x-ray emitter and x-ray detector projected onto the patient support, since artifact-free reconstruction algorithms are known for an arrangement of this type. The fade-in facility is advantageously arranged to be rotatable such that the x-ray beam can be adjusted to a sloping region to be examined. This can be attributed to a tilt of the examination object to a support positioned between the x-ray emitter and x-ray detector, and below the aperture, or to the anatomy of the examination object. The use of an aperture designed as a slit allows the radiation exposure for the examination object to be reduced.

In a further advantageous embodiment of the invention, the aperture is designed as a rectangle. This allows a further reduction in the radiation exposure to be achieved. The x-ray beam is restricted in two directions which are orthogonal to one another by means of a rectangular aperture. The diaphragm elements restricting the aperture are affixed to the fade-in facility in a moveable fashion, so that the diaphragm elements can be adjusted manually or by means of corresponding other regulating means, in order to adjust the beam limiting surface to the region to be examined. The examination object is thus exposed to the lowest possible radiation exposure. The rectangular, adjustable diaphragm is, similar to the circular, adjustable diaphragm, suited to enabling a restriction of the x-rays, which only record individual organs to be examined or their partial areas. Hence, this foregoes the need during examinations, e.g. on the beating heart, to expose the whole thorax to the x-rays, in the case of a plurality of projection data acquisitions. The x-ray can instead be adjusted to the projected organ size of the respective projection direction so that a spatial display can be determined with the aid of a plurality of projection data sets from different projection directions.

In a further advantageous embodiment of the invention, means are provided for adjusting the aperture and/or the aperture distance, which are effectively connected to a facility for controlling the regulating means. The regulating means can be designed for instance as a drive facility and/or as a drive. The drive facility, for instance an electric motor, generates a force which is transmitted by a drive on the adjustable diaphragm elements as a linear or circular motion. This type of regulating means allows a more rapid and more precise adjustment of the aperture. A control facility allows the aperture to be controlled such that the restriction of the x-ray beam is always adjusted to the region to be examined by means of the aperture provided. A control facility thus allows a spatial display of a region to be examined to be determined in a time-efficient manner by means of a controlled diaphragm, with the radiation exposure being significantly reduced for the examination object.

In a preferred embodiment of the invention, a unit for inputting/outputting target and/or actual values of control parameters and connected to the control facility is provided. The user is thus able to select apertures for instance, predefine the distance of the x-ray ray source from the fade-in facility and predetermine the region to be examined etc. Furthermore, the input/output unit can display the examination progress and the actual and target values of a control parameter.

The region to be examined by the medical person can be selected by means of markers, which are positioned on the examination object at the borders of the region to be examined. The position of a marker can be recorded by means of a localization system. The control facility is then fed the recorded position of the existing markers. The known position of the diaphragm and the x-ray source as well as the position and number of markers allows the aperture to be adjusted by the regulating means controlled by the control facility such that the x-ray beam does not touch the markers directly for instance or is directly surrounded therewith. If a slit is used as an aperture for instance, two markers are affixed to the examination object, between which the region to be examined of the examination object is arranged, according to the medical person skilled in the art. The controller adjusts the slit width by means of the regulating means such that precisely the region of the examination object lying between the two markers is radiated with the x-ray beam. To determine a region to be examined by means of locatable markers, a different number of markers can be provided for different shapes of the aperture. In order to adjust a rectangular diaphragm, four locatable markers can be provided for instance, whereas only one marker is provided for a circular diaphragm.

### BRIEF DESCRIPTION OF THE DRAWINGS

Further advantages of the invention result from an exemplary embodiment, which is described in more detail below with reference to the drawings, in which;

FIG. 1 shows a schematic representation of a side view of an inventive apparatus for spatial displays designed as an angiography device.

FIG. 2 shows a schematic representation of a front view of the angiography apparatus from FIG. 1,

FIG. 3 shows a schematic representation of a top view onto a fade-in facility of the angiography apparatus

### DETAILED DESCRIPTION OF THE INVENTION

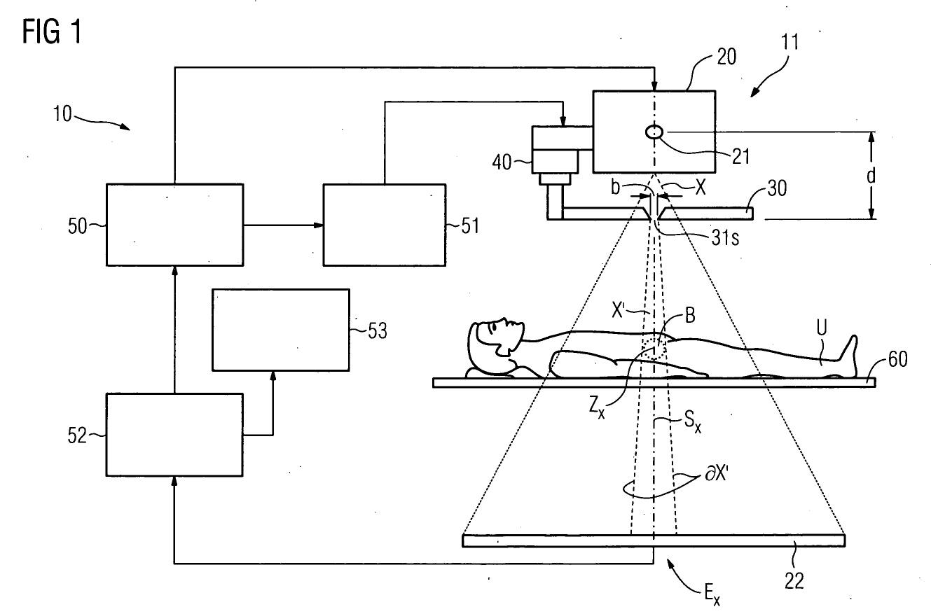

FIG. 1 shows a device for spatially displaying a region B to be examined of an examination object in the form of an angiography apparatus 10. The angiography apparatus 10 features an x-ray recording facility 11, which comprises an x-ray emitter 20, in which an x-ray source 21 is permanently arranged. An x-ray detector 22 is arranged opposite the x-ray emitter 20, said x-ray detector 22 detecting an x-ray beam arriving at the x-ray detector 22. The x-ray emitter 20 and the x-ray detector 22 cannot be moved relative to one another. However, the recording facility 11 is mounted in a rotatable manner about an examination object U which can be positioned on a support 60, said examination object U being arranged between the x-ray emitter 20 and the x-ray detector 22.

The x-ray source 21 generates an x-ray beam X, which is essentially radiated in the direction of an x-ray detector 22, and comprises a beam centerline Sₓ. If an examination object U is arranged on the support 60 between the x-ray emitter 20 and the x-ray detector 22, the examination object U is x-rayed by an x-ray beam X when the x-ray source 21 is operating. The arrangement of the x-ray emitter 20 and the x-ray detector 22 can be designed here in a different manner, e.g. as an upper table system, as shown in FIG. 1, or also as a lower table system (not shown). The recording facility 11 comprises a fade-in facility 30, which is arranged between the examination object U and the x-ray source 21. The fade-in facility 30 serves to fade the x-ray beam X generated by the x-ray source 21 into a split beam X′. To this end, the fade-in facility 30 features an aperture 31s, which is designed as a slit. The fade-in facility 30 is essentially manufactured from x-ray-absorbent material, in this case lead. The slit 31s features a slit width b, which can be adjusted by means of regulating means in the form of diaphragm elements (not shown). In addition, the slit features a slit length which does not however restrict the expansion of the x-ray beam X emitted by the x-ray source 21, said expansion being predetermined by a beam limiting surface. A distance d is provided between the x-ray source 21 and the fade-in facility 30. This distance d of the slit 31s from the x-ray source 21 can be adjusted here by means of a telescope arm 40. The adjustment of the distance d and the slit width b can be carried out manually or automatically.

The slit 31s is arranged between the x-ray source 21 and the examination object U such that the beam centerline Sₓ passes through the slit 31s in a point symmetrical manner. The x-ray beam X emitted by the x-ray source 21 is faded-in through the gap 31s in a wedge-shaped fashion onto a split beam X′. A configuration featuring two essentially flat partial areas ∂X′ restricting the x-ray beam is caused by fading-in the x-ray beam X onto the split beam X′. These partial areas ∂X′ of the beam limiting surface are inclined towards one another. The adjustments of the fade-in facility 30, which comprise the distance d and the slit width b, are adjusted such that the partial areas ∂X′ Of the beam limiting surfaces rest closely against the region B to be examined.

In the present case, a human stomach B of a child U is to be examined. To keep the radiation exposure for the child U as minimal as possible, the x-ray emitter 20 is arranged above the child U positioned on the support such that the smallest possible slit width b of the fade-in facility 30 can be selected, in order to allow both partial areas ∂X′, of the beam limiting surfaces to rest closely against the stomach B. The slit width b of the fade-in facility 30 can also be selected with regard to the age, size and stature of the child U.

A data processing system 52 is used to create a spatial display of the stomach B of the child U. The plurality of two-dimensional projection data sets of the stomach B recorded by means of the faded-in split beam X′ is fed to the data processing unit 52 and is stored there. A spatial representation of the stomach B is determined from the plurality of two-dimensional projection data sets, by means of a reconstruction method, said spatial display being shown on an input/output unit 53.

The fade-in facility 30 can be adjusted by way of regulating means, in this instance a drive facility 51. The adjustment encompasses the slit width b of the fade-in facility 30 as well as its distance from the x-ray source 21. The size of these parameters can be predefined by way of an input/output unit 53. This can be carried out by directly inputting values, or instead by marking the region to be examined on a patient model output to the input/output device 53 and present in the data processing system 52. By marking the region B to be examined on the patient model, the fade-in facility 30 is adjusted by the control facility 50 in conjunction with the regulating means 51 such that the partial areas ∂X′ of the beam limiting surface of the x-ray beam X′ rest closely against the region B to be examined.

While recording the two-dimensional projection data sets, the x-ray emitter 20 and the x-ray detector 22 facing the x-ray emitter 20 are rotated about the region B to be examined. While rotating about the stomach B, projection data sets of stomach B are recorded at specific time intervals. The projection direction of the projection data sets determined by the beam centerline Sₓ of the faded-in x-ray split beam X′ lies in one of the examination planes Ex penetrating the stomach B, said examination plane being vertical to the sheet plane in FIG. 1 and the vertical projection of which coincides on the sheet plane with the beam centerline Sₓ indicated in FIG. 1. The beam centerline Sₓ of the recorded projection data sets form concurrent lines with a common point. The common point is in this way identical to an examination center Zₓ. The examination center Zₓ is intersected by all beam centerlines Sₓ of the faded-in x-ray split beam X′ when the recording facility 11 is rotated about the examination object U. Expediently, the examination center Zₓ lies in the center of the region B to be examined, i.e. in this case in the central point of the stomach. This ensures the best possible imaging of the region B to be examined in the projection data set and thus in the spatial display to be determined.

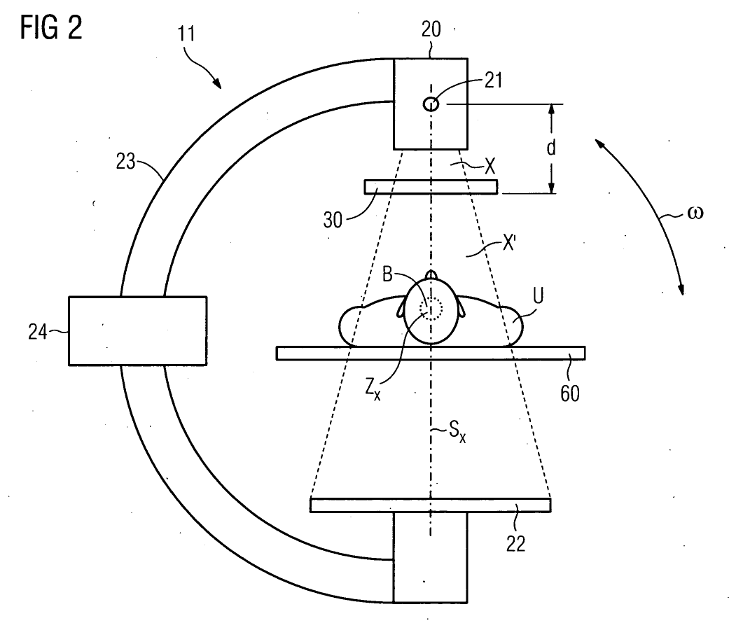

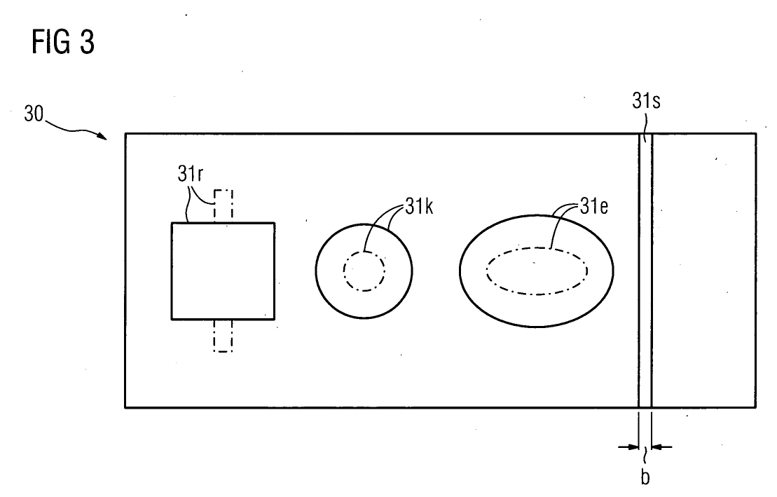

FIG. 2 shows a front view of the recording facility 11 illustrated schematically in FIG. 1. The x-ray emitter 20 with the x-ray source 21 is connected to the x-ray detector 22 with a C-arm 23. The C-arm 23 is affixed to a stand (not shown). To enable a controlled rotation of the x-ray emitter 20 and the x-ray detector 20 about the examination object U by means of the control device 50 illustrated in FIG. 1, a drive facility 24, which is designed as an orbital drive system 24, is provided. The orbital drive system 24 rotates the recording facility 11 at an angular speed ω about the examination object U. The rotation of the recording facility 11 comprises two top dead centers, between which the recording facility 11 is rotated. Furthermore, FIG. 2 illustrates that with the fade-in facility 30 designed as a slit 31s, the whole width of the child is x-rayed using the faded-in split beam X′, which features the same spatial dimensions in this direction as the x-ray beam X. A layer of the child U is thus radiated. By using an adjustable aperture 31r designed as a rectangle, see FIG. 3, the beam limiting surface shown in FIG. 2 can also be adjusted in the direction of the region B to be examined such that this beam limiting surface also closely surrounds the stomach B, thereby not x-raying the whole width of the child U.

FIG. 3 shows a fade-in facility 30, comprising different adjustable apertures. In particular, in addition to a slit 31s, the fade-in facility 30 comprises an adjustable rectangular aperture 31r, a circular aperture 31k which can be adjusted using an iris diaphragm (not shown) as well as an ellipsoid, adjustable aperture 31e. As a function of the shape and size of the region B to be examined of the examination object U, a corresponding diaphragm can be selected and adjusted to the region to be examined. The dashed and drawn-through markers for the respective apertures 31s or 31r or 31e or 31k within the fade-in facility 30 show two of several possible control positions of the respective apertures 31s or 31r or 31e or 31k. A fade-in facility 30 of this type does not require a manual exchange of diaphragms with associated apertures. A drive facility, for instance the drive facility 51 mentioned in FIG. 1, allows the apertures 31s or 31r or 31e or 31k arranged on the fade-in facility 30 to be moved and adjusted in the radiation path of the emitted x-ray X, see FIG. 1, such that the diaphragm elements restricting the x-ray beam X generate a beam limiting surface ∂X′, see FIG. 1, which closely surrounds the region B to be examined, see FIG. 1.

1.-10. (canceled)

11. A method for spatially displaying a region to be examined of an examination object in a medical procedure, comprising:
penetrating the examination object in a projection direction with an x-ray beam comprising a beam centerline extending in the projection direction;
restricting the x-ray beam by a beam limiting surface;
adjusting the x-ray beam as a function of the region to be examined such that the beam limiting surface closely surrounds the region to be examined;
recording a two-dimensional projection data set of the region to be examined in the projection direction from the penetrated x-ray beam;
determining a three-dimensional image data set of the region to be examined from the two-dimensional projection data set by an image reconstruction method; and
displaying the three-dimensional image data set of the region to be examined.

12. The method as claimed in claim 11, wherein the x-ray beam is wedge shaped so that two essentially flat partial areas of the beam limiting surface that are inclined towards one another locate closely on the region to be examined.

13. The method as claimed in claim 11, wherein the x-ray beam is pyramid shaped so that two essentially flat partial areas of the beam limiting surface that are inclined towards one another locate closely on the region to be examined.

14. The method as claimed in claim 11, wherein the beam centerline passes through a common examination center located in the region to be examined.

15. The method as claimed in claim 11, wherein the projection data sets is recorded with a constant beam setting.

16. The method as claimed in claim 11, wherein the x-ray beam is adjusted by fading the x-ray beam into a split beam.

17. A device for displaying a region to be examined of an examination object in a medical procedure, comprising:
an x-ray emitter that is rotated about the examination object in order to generate an x-ray beam;
an x-ray detector that is rotated about the examination object in order to record a part of the x-ray beam penetrating the examination object; and
a fade-in device arranged at a distance from the x-ray emitter, wherein the fade-in device comprises an aperture that fades in the x-ray beam into a split beam and the aperture is adjusted as a function of the region to be examined so that the split beam closely surrounds the region to be examined.

18. The device as claimed in claim 17, wherein the aperture is selected from the group consisting of: a slit, a rectangle, a circle, and an ellipsoid.

19. The device as claimed in claim 17, wherein the distance is adjusted as a function of the region to be examined.

20. The device as claimed in claim 19, further comprising a drive unit that adjusts the aperture or the distance.

21. The device as claimed in claim 20, wherein the drive unit is controlled by a control unit.

22. The device as claimed in claim 21, wherein the control unit is connected to an input/output unit that inputs or outputs a target or an actual value of a control parameter.

23. The device as claimed in claim 22, wherein the control parameter is a size of the aperture or the distance.

24. The device as claimed in claim 17, wherein the fade-in device comprises an x-ray absorbent material.

25. A device for displaying a region to be examined of an examination object in a medical procedure, comprising:
an x-ray emitter that is rotated about the examination object in order to generate an x-ray beam;
an x-ray detector that is rotated about the examination object in order to record a part of the x-ray beam penetrating the examination object; and
a fade-in device arranged at a distance from the x-ray emitter, wherein the fade-in device comprises an aperture that fades in the x-ray beam into a split beam and the distance is adjusted as a function of the region to be examined so that the split beam closely surrounds the region to be examined.

26. The device as claimed in claim 25, wherein the aperture is adjusted as a function of the region to be examined.
